(19) United States
(12) Patent Application Publication
(10) Pub. No.: US 2018/0243544 A1
(43) Pub. Date: Aug. 30, 2018

(54) MEDICAL DEVICE FOR GENERATING ULTRASONIC WAVES AND ELECTRICAL STIMULATION SIGNAL

(71) Applicants: Seong Seok YANG, Seongnam-si, Gyeonggi-do (KR); GENEMEDI Co., LTD., Cheonan-si, Chungcheongnam-do (KR)

(72) Inventor: Seong Seok YANG, Seongnam-si (KR)

(21) Appl. No.: 15/752,693

(22) PCT Filed: Aug. 17, 2016
(86) PCT No.: PCT/KR2016/009034

§ 371 Date: Feb. 14, 2018

(30) Foreign Application Priority Data
Aug. 20, 2015 (KR) 10-2015-0117397

(51) Int. Cl. A61M 37/00; A61N 1/36; B06B 1/02; B06B 1/06

(52) U.S. Cl. CPC A61M 37/0092; A61N 1/36014; B06B 1/0207; H01L 41/1876; A61M 2205/054; B06B 2201/76; B06B 1/0662

(57) ABSTRACT

A medical device can comprise: a piezoelectric element for generating ultrasonic waves; a transmission unit coming into contact with the piezoelectric element, and transmitting, to the skin, the ultrasonic waves and an electrical stimulation signal generated from the outside; and a guide having a ring shape having an inner surface and an outer surface, and coming into contact with one surface of the transmission unit in order to provide a functional material accommodation space. The functional material accommodation space can be defined by the inner surface of the guide and the one surface of the transmission unit. According to the present invention, a functional material accommodation space capable of maximizing the area in which a functional material and the skin make contact can be provided, and the configuration of a circuit for generating ultrasonic waves and the configuration of a circuit for generating an electrical stimulation signal can be simplified.

### CROSS-REFERENCE TO RELATED APPLICATIONS

This U.S. non-provisional patent application claims priority under 35 U.S.C. § 119 of Korean Patent Application No. 10-2015-0117397, filed on Aug. 20, 2015, the entire contents of which are hereby incorporated by reference.

### BACKGROUND

The present disclosure herein relates to a medical device, and more particularly, to a medical device generating an ultrasonic wave for effectively transferring a functional material into a human body and an electrical stimulation signal for treatment effects.

There are various methods for effectively injecting a functional material for beauty care or treatment purposes into a skin. However, since a human skin has a resistance force with respect to foreign substances, the efficiency of the injection is limitedly enhanced. Accordingly, an iontopheresis technique using an electric repulsive force or a phonophoresis technique using an ultrasonic wave have been used to effectively inject a functional material into a skin.

The iontopheresis technique uses a method of applying a functional material having an electric charge, which is intended to be injected into a body, to a skin. To this end, the functional material is injected into the human body in such a manner that an electrode plate having the same electric charge as that of the functional material is attached to the skin on which the functional material is spread, and an electrode plate having an opposite electric charge is attached to another portion of a human body.

The phonophoresis technique uses a function forming micro bubble to inject the functional material into a body while temporarily reducing a resistance of the skin. To this end, the functional material is spread to the skin and then rubbed by using a device generating an ultrasonic wave. However, when a general ultrasonic device is used, a high power ultrasonic wave is used to increase the efficiency of injecting the functional material into the skin, thereby occasionally generating heat in the human body. The generated heat may result in death of skin cells. Meanwhile, when the power of the ultrasonic wave is lowered to prevent the death of cells, the efficiency of injecting the functional material into a skin may decrease.

Also, when the functional material is injected into a body by using the method of directly applying the functional material to the skin and then scrubbing the same, maintaining a constant thickness of the functional material spread on the skin is another problematic issue. This is a factor reducing the efficiency of injecting the function material into the skin.

Meanwhile, when a separated storage space for storing the functional material is defined in the ultrasonic wave device, the ultrasonic wave may be reflected and distorted due to the structure of the storage space for storing the functional material. This is also a factor reducing the efficiency of injecting the function material into the skin. Thus, it is very important to configure the functional material storage space capable of minimizing the reflection and distortion of the ultrasonic wave while maintaining the thickness of the functional material contacting the skin.

### SUMMARY

The present disclosure provides a medical device generating an ultrasonic wave for effectively injecting a functional material into a skin and an electrical stimulation signal for treatment effects.

An embodiment of the inventive concept provides a medical device including: a piezoelectric element configured to generate an ultrasonic wave; a transmission unit configured to transmit the ultrasonic wave and an electrical stimulation signal generated from the outside to a skin while contacting the piezoelectric element; and a guide having a ring shape having an inner side of and an outer side and contacting one surface of the transmission unit in order to define a functional material accommodation space. The functional material accommodation space is defined by the inner side of the guide and the one surface of the transmission unit.

In an embodiment of the inventive concept, a medical device includes: an electric stimulation controller configured to generate an electrical stimulation signal; and an ultrasonic wave generating and transmitting element including a piezoelectric element configured to generate an ultrasonic wave under control of an ultrasonic wave controller and a housing defined in a depth direction to accommodate a functional material. The ultrasonic wave generating and transmitting element is configured to transfer the functional material provided in the housing to the skin by using the ultrasonic wave, the ultrasonic wave generating and transmitting element is configured to transmit the electrical stimulation signal to the skin, and the housing has a constant inner diameter along the depth direction.

In an embodiment of the inventive concept, a medical device includes: an electric stimulation controller configured to generate a first electrical stimulation signal having a first polarity and a second electrical stimulation signal having a second polarity; a first ultrasonic wave generating and transmitting element including a first piezoelectric element configured to generate an ultrasonic wave under control of an ultrasonic wave controller and a first housing defined in a depth direction to accommodate a functional material; and a second ultrasonic wave generating and transmitting element including a second piezoelectric element configured to generate the ultrasonic wave under the control of the ultrasonic wave controller and a second housing defined in the depth direction to accommodate the functional material. The first ultrasonic wave generating and transmitting element and the second ultrasonic wave generating and transmitting element are configured to transmit the functional material provided in the first housing and the second housing to the skin in a respective manner, the first ultrasonic wave generating and transmitting element is configured to transmit the first electrical stimulation signal to the skin through the first housing, and the second ultrasonic wave generating and transmitting element is configured to transmit the second electrical stimulation signal to the skin through the second housing, and each of the first housing and the second housing has a constant inner diameter along the depth direction.

### BRIEF DESCRIPTION OF THE FIGURES

FIG. 1 is a view exemplarily illustrating a medical device according to an embodiment of the inventive concept.

FIG. 2 is a view exemplarily illustrating an ultrasonic wave generating and transmitting element in FIG. 1 according to an embodiment of the inventive concept.

FIG. 3 is a view exemplarily illustrating a medical device according to an embodiment of the inventive concept.

FIG. 4A and 4B are schematic graphs showing waveforms of an ultrasonic wave and an electrical stimulation signal, which are generated by the medical device in FIGS. 2 and 3.

FIG. 5A and 5B are schematic graphs showing waveforms of an ultrasonic wave and an electrical stimulation signal, which are generated by the medical device in FIGS. 2 and 3.

FIG. 6A to 6F are views exemplarily illustrating various shapes of a guide of a medical device according to an embodiment of the inventive concept.

FIG. 7 is a view exemplarily illustrating the ultrasonic wave generating and transmitting element in FIG. 1 according to an embodiment of the inventive concept.

FIG. 8 is a view exemplarily illustrating a medical device according to an embodiment of the inventive concept.

FIG. 9 is a view exemplarily illustrating a medical device according to an embodiment of the inventive concept.

FIG. 10A to 10C are schematic graphs showing waveforms of an ultrasonic wave and an electrical stimulation signal, which are generated by the medical device in FIG. 9.

FIG. 11 is a view exemplarily illustrating a medical device according to an embodiment of the inventive concept.

### DETAILED DESCRIPTION

Hereinafter, embodiments of the inventive concept are exactly and precisely described so that the present disclosure may be easily implemented by those with ordinary skill in the technical field of the present invention.

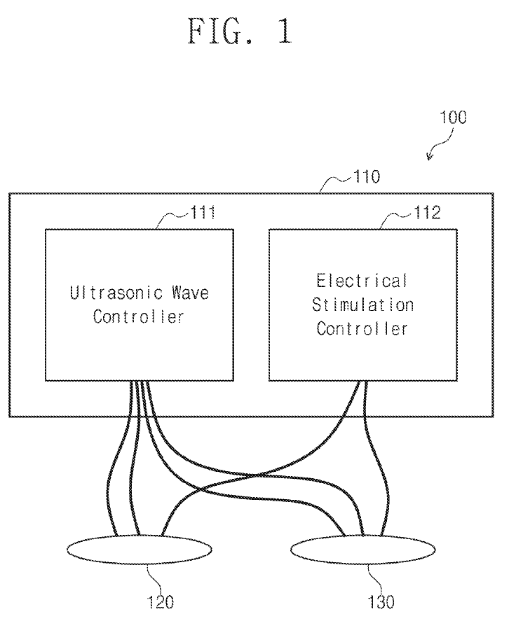

FIG. 1 is a view exemplarily illustrating a medical device according to an embodiment of the inventive concept. Referring to FIG. 1, a medical device 100 may include a controller 110, a first ultrasonic wave generating and transmitting element 120, and a second ultrasonic wave generating and transmitting element 130. Although the medical device 100 includes two ultrasonic wave generating and transmitting elements 120 and 130 in this embodiment, the medical device 100 may be realized to include one ultrasonic wave generating and transmitting element depending on an embodiment.

The medical device 100 may be configured to inject a functional material into a skin by using an ultrasonic wave. For this configuration, the controller 110 may include an ultrasonic wave controller 111 controlling to generate an ultrasonic wave. Under the control of the ultrasonic wave controller 111, a piezoelectric element (not shown) included in each of the ultrasonic wave generating and transmitting elements may generate an ultrasonic wave. The generated ultrasonic wave allows a functional material provided to a functional material accommodation space (not shown) or a housing (not shown) provided in each of the ultrasonic wave generating and transmitting elements 120 and 130 to be effectively transmitted into a body below a skin.

For the effective transmission of the functional material, each of the ultrasonic wave generating and transmitting elements 120 and 130 may include the functional material accommodation space or the housing (not shown). The functional material accommodation space (or housing) may be defined in one surface of each of the ultrasonic wave generating and transmitting elements 120 and 130 so that a surface area, which contacts the skin, of the functional material is maximized. The specific structure of the functional material accommodation space will be described in more detail later with reference to FIG. 2.

The medical device 100 may be configured to transmit an electrical stimulation signal generated by an electric stimulation controller 112 to the skin. For this configuration, each of the ultrasonic wave generating and transmitting elements 120 and 130 may include a conductive material. For example, the generated electrical stimulation signal may be transmitted to the skin through the ultrasonic wave generating and transmitting elements 120 and 130, and the electrical stimulation signal may be used for a physical treatment, an inflammation treatment, and a pain treatment.

For example, the electric stimulation controller 112 may generate a positive electrical stimulation signal and a negative electrical stimulation signal. For example, the electric stimulation controller 112 may transmit the positive electrical stimulation signal to the first ultrasonic wave generating and transmitting element 120 and the negative electrical stimulation signal to the second ultrasonic wave generating and transmitting element 130.

However, an embodiment of the inventive concept is not limited to the method for generating electrical stimulation signals by the electric stimulation controller 112. For example, the electric stimulation controller 112 may alternately generate a positive electrical stimulation signal and a negative electrical stimulation signal. Thereafter, the positive electrical stimulation signal and the negative electrical stimulation signal, which are alternately generated, may be transmitted to the ultrasonic wave generating and transmitting elements 120 and 130. In this case, the electric stimulation controller 112 may variously adjust a frequency at which the positive electrical stimulation signal and the negative electrical stimulation signal are generated. In addition, the electric stimulation controller 112 may adjust an intensity of the electrical stimulation signal.

Meanwhile, the ultrasonic wave controller 111 may control the ultrasonic wave generating and transmitting elements 120 and 130 so that an ultrasonic wave has an active interval and an inactive interval in a repeated manner. That is, a reference frequency of an ultrasonic wave may be a sum of a time of the active interval and a time of the inactive interval. Also, the electric stimulation controller 112 may not generate an electrical stimulation signal in the active interval of an ultrasonic wave. Also, the electric stimulation controller 112 may generate an electrical stimulation signal in the active interval of the ultrasonic wave.

The medical device 100 may be configured to receive a power from the outside to generate an ultrasonic wave and an electrical stimulation signal. Also, the medical device 100 may include a detachable battery module to be easily carried out. Also, the medical device 100 may include both of the configuration receiving a power from the outside and the battery module or may include only the battery module without including the configuration receiving a power from the outside.

As described above, the medical device 100 according to an embodiment of the inventive concept is simply described. Through the above-described configuration, the functional material containing a medicine constituent and a cosmetic constituent (e.g., in a liquid state type such as lotion and gel) may be effectively transferred to a skin by an ultrasonic wave. In addition, the electrical stimulation signal for a physical treatment or the like may be transferred to the skin. Accordingly, the treatment effect of the medical device 100 may be improved. Also, as the ultrasonic wave controller 111 controlling the ultrasonic wave generation and the electric stimulation controller 112 are configured as a separated circuit, and the ultrasonic wave and the electrical stimulation signal are alternately generated, the configuration of the circuit (i.e., controller 110) may be simplified.

FIG. 2 is a view exemplarily illustrating the ultrasonic wave generating and transmitting element 120 in FIG. 1. For example, the ultrasonic wave generating and transmitting elements 120 and 130 in FIG. 1 may have the substantially same configuration as each other. The ultrasonic wave generating and transmitting element 120 may include a piezoelectric element 121, a transmission unit 122, and a guide 123.

The piezoelectric element 121 may include various piezoelectric materials such as lead zirconium titanate (PZT), barium titanate, ammonium dihydrogen phosphate, and ethylenediamine tartrate. However, these materials are illustrative, and various other piezoelectric materials may be used. The piezoelectric element 121 may generate an ultrasonic wave under the control of the ultrasonic wave controller 111. For example, the piezoelectric element 121 may generate an ultrasonic wave to have an active interval and an inactive interval in a repeated manner.

The transmission unit 122 may transmit the ultrasonic wave generated by the piezoelectric element 121 to the functional material accommodation space. The ultrasonic wave allows the functional material provided in the functional material accommodation space to be effectively transferred to the skin. The transmission unit 122 may receive the electrical stimulation signal generated by the electric stimulation controller 112 to transmit the received signal to the skin. For example, the electrical stimulation signal may be transmitted to the skin through a portion contacting the skin. For example, the transmission unit 122 may include a conductive material.

The guide 123 may contact a portion of one surface A of the ultrasonic generating and transmitting element 120. For example, the guide 123 may have a ring shape having an inner side and an outer side. The inner side of the guide 123 is indicated by B in FIG. 2. As the guide 123 contacts a portion of one surface of the ultrasonic generating and transmitting element 120, the functional material accommodation space may be defined by the inner side of the guide 123 and the one surface A of the ultrasonic wave generating and transmitting element 120. In this specification, the functional material accommodation space may be referred to as a housing. That is, the housing may be defined in a depth direction of the ultrasonic wave generating and transmitting element 120, and the depth of the housing may be ‘h’ as illustrated in FIG. 2. For example, the depth h of the housing may be about 0.05 mm to about 2.0 mm. For example, when the functional material provided to the housing is the medicine material, the depth h of the housing may be about 2.0 mm. For example, the housing may have a constant inner diameter along the depth direction.

As the guide 123 is attached to one surface of the ultrasonic wave generating and transmitting element 120 to define the functional material accommodation space (i.e., housing), a relatively uniform amount of the functional material may be accommodated. In addition, through the above-described structure, the functional material (not shown) provided to the functional material accommodation space may have a maximized surface area contacting the skin. The functional material provided to the functional material accommodation space may be injected into the skin by the ultrasonic wave generated from the piezoelectric element 121. In addition, the electrical stimulation signal generated by the electric stimulation controller 112 may be transmitted to the skin contacting the guide 123 through the transmission unit 122 and the guide 123. For example, the ultrasonic wave may be generated in a section during which the electrical stimulation signal is not generated, and the electrical stimulation signal may be generated in a section during which the ultrasonic wave is not generated.

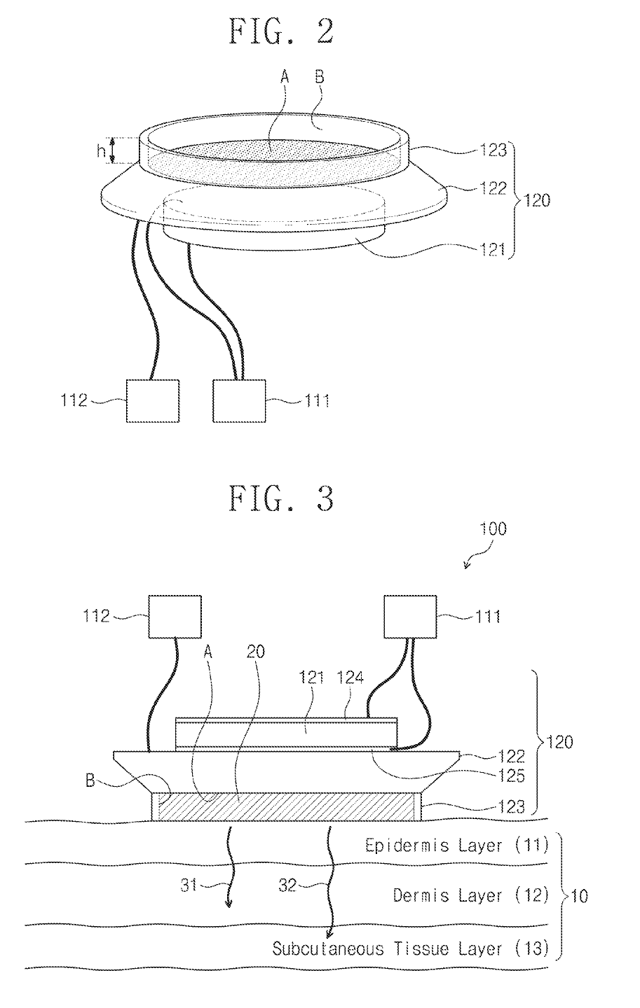

FIG. 3 is a view exemplarily illustrating the medical device according to an embodiment of the inventive concept. Referring to FIG. 3, the medical device 100 may include a ultrasonic wave controller 111, an electric stimulation controller 112, and an ultrasonic wave generating and transmitting element 120. The medical device 100 will be described with reference to FIG. 2 to help understanding of description.

The ultrasonic wave generating and transmitting element 120 may include a piezoelectric element 121, electrode films 123 and 125, a transmission unit 122, and a guide 123. The electrode films 124 and 125 may be provided on two surfaces of the piezoelectric element 121, respectively. For example, the electrode films 124 and 125 may be provided on two surfaces of the piezoelectric element 121, respectively. For example, the first electrode film 124 may be provided on a surface, which contacts the transmission unit 122, of the piezoelectric element 121, and the second electrode film 125 may be provided on a surface, which is not in contact with the transmission unit 122, of the piezoelectric element 121.

The ultrasonic wave controller 111 may be electrically connected to the first electrode film 124 and the second electrode film 125. The ultrasonic wave controller 111 may be connected to the transmission unit 122 instead of being connected to the first electrode film 124. Also, the ultrasonic wave controller 111 may be connected to the transmission unit 122 instead of being connected to the second electrode film 125. The reason is that the piezoelectric element 121 and the conductive transmission unit 122 are electrically connected through the first electrode film 124. The ultrasonic wave controller 111 may control the piezoelectric element 121 so that the piezoelectric element 121 generates the ultrasonic wave having an active interval and an inactive interval.

The electric stimulation controller 112 may be electrically connected to the transmission unit 122. For example, the electric stimulation controller 122 may generate an electrical stimulation signal in the section during which the ultrasonic wave is inactivated and may not generate an electrical stimulation signal in the section during which the ultrasonic wave is activated. The electrical stimulation signal generated by the electric stimulation controller 122 may be transmitted to a skin 10 through the transmission unit 122 and the guide 123.

In general, the skin 10 may include an epidermis layer 11, a dermis layer 12, and a subcutaneous tissue layer 13. The functional material is necessarily transferred to the dermis layer 12 inside the skin 10 through the epidermis layer 11 (illustrated as an arrow 31 in FIG. 3) or transferred to the subcutaneous tissue layer 13 (illustrated as an arrow 32 in FIG. 3) so that the functional material makes effects. However, due to a resistant force of the epidermis layer 11 with respect to foreign substances, it may be difficult that the functional material is transferred to the dermis layer 12 or the subcutaneous tissue layer 13.

The ultrasonic wave generated by the piezoelectric element 121 may be used for phonophoresis. That is, as the permeability of a cell membrane is enhanced due to the thermal effect of the ultrasonic wave, the functional material may be transferred further deeply into the skin 10. Also, as the diffusion speed of the functional material increases due to the mechanical effect of the ultrasonic wave, the speed of the functional material transferred into the skin 10 may further increase.

The ultrasonic wave generated through the piezoelectric element 121 may generate a wavelength having various frequencies according to the kinds of the functional material 20 provided in the functional material accommodation space or the purpose of the medical device. To this end, the medical device 100 may support various kinds of operation modes capable of generating various frequencies. For example, the operation modes may be selected by a user. Also, the medical device 100 may include various kinds of user interfaces so that the user selects various kinds of operation modes.

For example, when a user of the medical device 100 wants to use the functional material 20 having a medical effect such as infection or pain relief, the piezoelectric element 121 may generate a frequency of about 1 MHz on the basis of the mode selection of the user. As a result, for example, the functional material 20 may be transferred to a depth of about 5 cm to about 7 cm from the surface of the skin 10. However, the above numerical value may be illustrative and varied according to the operation mode of the medical device 100.

For example, when the user of the medical device 100 wants to use the functional material 20 having a beauty care function such as skin care, the piezoelectric element 121 may generate a frequency of about 3 MHz on the basis of the mode selection of the user. As a result, for example, the functional material 20 may be transferred to a depth within about 1 cm from the surface of the skin 10. That is, the functional material and the ultrasonic wave energy may be concentrated on a partial area from the epidermis layer 11 to the dermis layer 12. Likewise, the above numerical value may be illustrative and varied according to the operation mode of the medical device 100.

FIGS. 4A and 4B are schematic graphs showing waveforms of the ultrasonic wave and the electrical stimulation signal, which are generated by the medical device in FIGS. 2 and 3, respectively. For example, FIG. 4A shows a schematic waveform of a ultrasonic wave, and FIG. 4B shows a schematic waveform of an electrical stimulation signal. Hereinafter, FIGS. 4A and 4B will be described together with reference to FIGS. 2 and 3 to help understanding of description.

Referring to FIGS. 4A and 4B, in an interval of t0 to t1, the ultrasonic wave generating and transmitting element 120 may not generate an ultrasonic wave. Meanwhile, in the interval of t0 to t1, the electric stimulation controller 112 may generate a positive electrical stimulation signal.

In an interval of t1 to t2, the ultrasonic wave generating and transmitting element 120 may generate an ultrasonic wave. Meanwhile, in the interval of t1 to t2, the electric stimulation controller 112 may not generate an electrical stimulation signal.

That is, the frequency of the ultrasonic wave may be a time between t0 to t2, and the ultrasonic wave may include an active interval and an inactive interval in one frequency. Likewise, that is, the frequency of the electrical stimulation signal may be a time between t0 to t2, and the electrical stimulation signal may include an active interval and an inactive interval in one frequency.

In an interval of t2 to t3, an interval of t3 to t4, and an interval of t4 to t5, an operation similar to the above may be implemented. That is, the electrical stimulation signal may not be generated in the interval during which the ultrasonic wave is generated, and the positive electrical stimulation signal may be generated in the interval during which the ultrasonic wave is not generated.

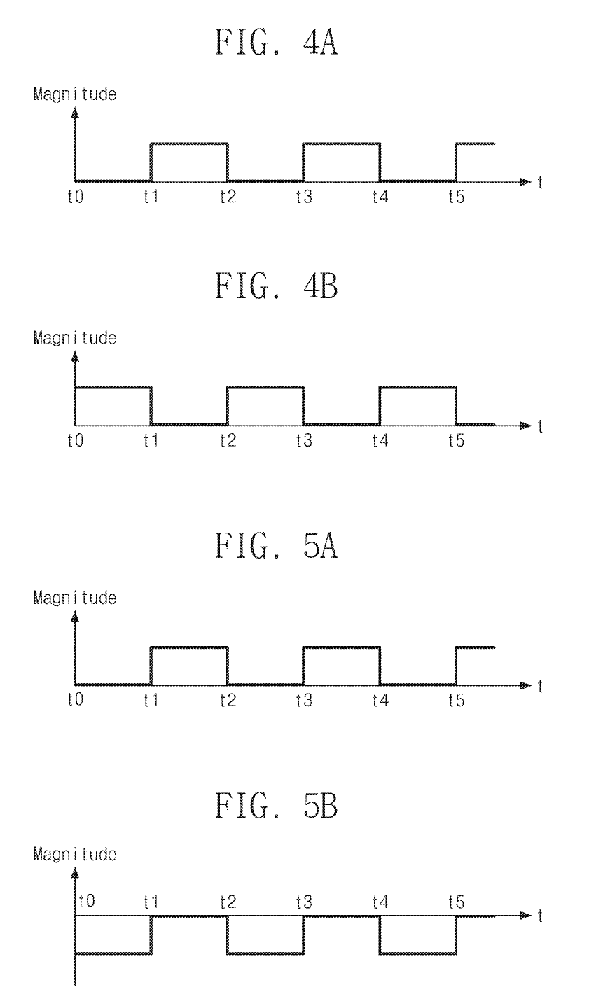

FIGS. 5A and 5B are schematic graphs showing waveforms of the ultrasonic wave and the electrical stimulation signal, which are generated by the medical device in FIGS. 2 and 3, respectively. For example, FIG. 5A shows a schematic waveform of the ultrasonic wave, and FIG. 5B shows a schematic waveform of the electrical stimulation signal. Hereinafter, FIGS. 5A and 5B will be described together with reference to FIGS. 2 and 3 to help understanding of description.

Referring to FIGS. 5A and 5B, in an interval of t0 to t1, the ultrasonic wave generating and transmitting element 120 may not generate an ultrasonic wave. Meanwhile, in the interval of t0 to t1, the electric stimulation controller 112 may generate a negative electrical stimulation signal.

In an interval of t1 to t2, the ultrasonic wave generating and transmitting element 120 may generate an ultrasonic wave. Meanwhile, in the interval of t1 to t2, the electric stimulation controller 112 may not generate an electrical stimulation signal.

In an interval of t2 to t3, an interval of t3 to t4, and an interval of t4 to t5, an operation similar to the above may be implemented. That is, the electrical stimulation signal may not be generated in the interval during which the ultrasonic wave is generated, and the negative electrical stimulation signal may be generated in the interval during which the ultrasonic wave is not generated.

As described above, the waveforms of the ultrasonic wave and the electrical stimulation signal, which are generated by the medical device according to an embodiment of the inventive concept through FIGS. 4A, 4B, 5A and 5B. However, the substantial waveform of the ultrasonic wave and the electrical stimulation signal, which are generated by the medical device according to an embodiment of the inventive concept in not limited to the square wave illustrated in FIGS. 4 A, 4B, 5A and 5B. For example, the ultrasonic wave generated by the medical device according to an embodiment of the inventive concept may have a wave shaped waveform, and the electrical stimulation signal may have a shape of a pulse or an impulse. The waveforms in FIGS. 4 A, 4B, 5A and 5B are intended to show that the ultrasonic wave and the electrical stimulation signal are alternately generated. An embodiment of the inventive concept is not limited to the embodiments in FIGS. 4 A, 4B, 5A and 5B.

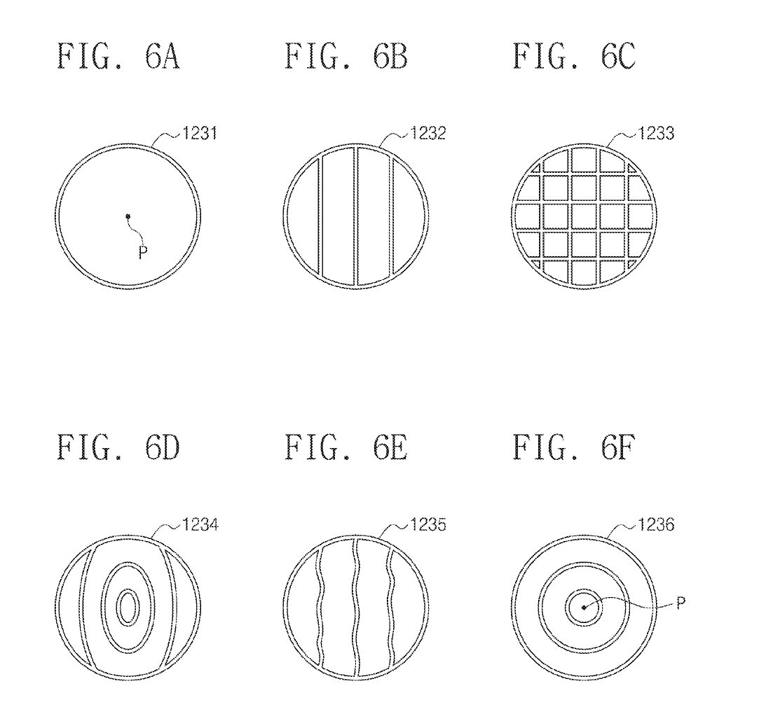

FIGS. 6A to 6F are views exemplarily illustrating various shapes of a guides of a medical device according to an embodiment of the inventive concept. For example, guides 1231 to 1236 in FIGS. 6A to 6F may be the guide 123 or deformed from the guide 123 in FIGS. 2 and 3. Hereinafter, FIGS. 6 A to 6F will be described together with reference to FIGS. 2 and 3 to help understanding of description.

The guide 1231 indicated in FIG. 6A may have a ring shape having a central point P with a constant radius. For example, the guide 1231 may have a constant diameter (i.e., diameter) along a depth direction of the functional material accommodation space.

The guide 1232 indicated in FIG. 6B may include a partition dividing the functional material accommodation space, which is defined when the guide 1232 contacts one surface A of the transmission unit 122, into a plurality of spaces. For example, according to the illustration of the drawing, the functional material accommodation space may be divided into four spaces by the partitions provided in the guide 1232.

The guides 1233 to 1236 indicated in FIGS. 6C to 6F are similar to the guide 1232 indicated in FIG. 6B in that each of the guides 1233 to 1236 indicated in FIGS. 6C to 6F includes a partition dividing the functional material accommodation space, which is defined when each thereof contacts one surface A of the transmission unit 122, into a plurality of spaces. Here, as illustrated in the drawings, only the shapes of the partitions are different. However, various shapes of the guides in the drawings are illustrative, and guides having other various shapes may be used.

FIG. 7 is a view exemplarily illustrating the ultrasonic wave generating and transmitting element in FIG. 1. An ultrasonic wave generating and transmitting element 220 may include a piezoelectric element 221, a transmission unit 222, and a guide 223. Since the structure and function of the piezoelectric element 221 and the transmission unit 222 are substantially the same as those described in FIG. 2, detailed description will be omitted.

The guide 223 may contact a portion of one surface A of the ultrasonic wave generating and transmitting element 220. For example, the guide 223 may have a ring shape having an inner side and an outer side. The inner side of the guide 223 is indicated by B in the drawing. As the guide 223 contacts one surface of the ultrasonic wave generating and transmitting element 220, a functional material accommodation space may be defined by the inner side B of the guide 223 and the one surface A of the ultrasonic wave generating and transmitting element 220. However, unlike the embodiment previously described in FIG. 2, the functional material accommodation space defined by the inner side B of the guide 223 and the one surface A of the ultrasonic wave generating and transmitting element 220 may have a bowl shape. The above functional material accommodation space may be referred to as a housing, and the housing may have an inner diameter gradually decreasing along a depth direction thereof.

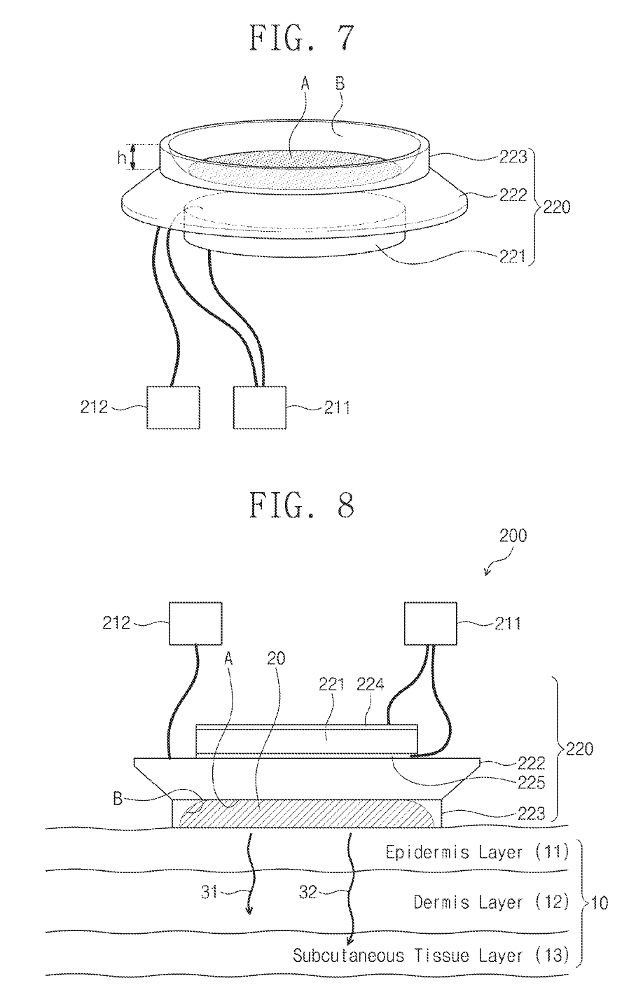

FIG. 8 is a view exemplarily illustrating a medical device 200 according to an embodiment of the inventive concept. Referring to FIG. 8, the medical device 200 may include an ultrasonic wave controller 211, an electric stimulation controller 212, and an ultrasonic wave generating and transmitting element 220. Since the medical device 200 that will be described in this drawing is substantially the same as the medical device described in FIG. 3 except for the shape of the guide, redundant description will be omitted. Hereinafter, FIG. 8 will be described together with reference to FIG. 7 to help understanding of description.

An ultrasonic wave generating and transmitting element 220 may include a piezoelectric element 221, a transmission unit 222, and a guide 223. The guide 223 may contact one surface of the transmission unit 222. As a result, a functional material accommodation space may be defined by one surface A of the transmission unit 222 and an inner side B of the guide 223. The functional material accommodation space in this embodiment may have a bowl shape instead of having the functional material accommodation space described in FIG. 3. That is, since the functional material accommodation space has a gentle slope, the functional material remained in the functional material accommodation space may be easily removed after a user uses the medical device 200.

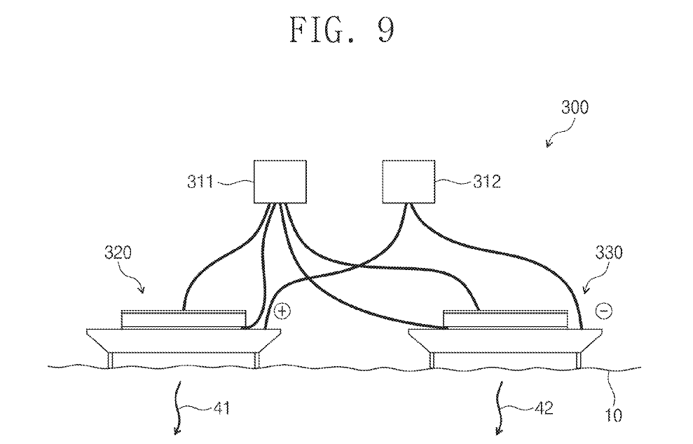

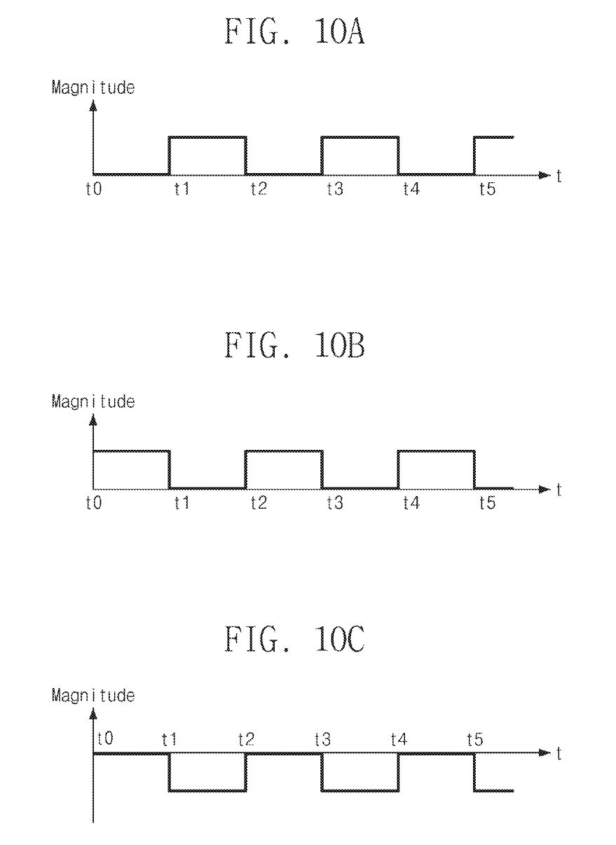

FIG. 9 is a view exemplarily illustrating a medical device 300 according to an embodiment of the inventive concept. The medical device may include an ultrasonic controller 311, an electric stimulation controller 312, a first ultrasonic wave generating and transmitting element 320, and a second ultrasonic wave generating and transmitting element 330. The first ultrasonic wave generating and transmitting element 320 and the second ultrasonic wave generating and transmitting element 330 may be one of the ultrasonic wave generating and transmitting elements previously described in FIGS. 2, 3, 7, and 8.

Referring to FIG. 9, the first ultrasonic wave generating and transmitting element 320 may generate an ultrasonic wave. For example, the ultrasonic wave controller 311 may be connected to electrode films provided on both surfaces of the piezoelectric element of the first ultrasonic wave generating and transmitting element 320, respectively, and may control the piezoelectric element to generate an ultrasonic wave. The ultrasonic wave generated by the piezoelectric element of the first ultrasonic wave generating and transmitting element 320 may allow the functional material provided in the functional material accommodation space to be transferred deeply into the skin 10 along a direction 41.

The first ultrasonic wave generating and transmitting element 320 may transmit an electrical stimulation signal generated by the electric stimulation controller 312 to the skin 10. For example, the electric stimulation controller 312 may generate a positive electrical stimulation signal, and the positive electrical stimulation signal may be transmitted to the skin 10 through the first ultrasonic wave generating and transmitting element 320.

The second ultrasonic wave generating and transmitting element 330 may generate an ultrasonic wave. For example, the ultrasonic wave controller 311 may be connected to electrode films provided on both surfaces of the piezoelectric element of the second ultrasonic wave generating and transmitting element 330, respectively, and may control the piezoelectric element to generate an ultrasonic wave. The ultrasonic wave generated by the piezoelectric element of the second ultrasonic wave generating and transmitting element 330 may allow the functional material provided in the functional material accommodation space to be transferred deeply into the skin 10 along a direction 42.

The second ultrasonic wave generating and transmitting element 330 may transmit an electrical stimulation signal generated by the electric stimulation controller 312 to the skin 10. For example, the electric stimulation controller 312 may generate a negative electrical stimulation signal, and the negative electrical stimulation signal may be transmitted to the skin 10 through the second ultrasonic wave generating and transmitting element 330.

FIGS. 10A, 10B, and 10C are schematic graphs showing waveforms of an ultrasonic wave and an electrical stimulation signal, which are generated by the medical device 300 in FIG. 9. For example, FIG. 10A shows a schematic waveform of an ultrasonic wave. FIG. 10B shows a schematic waveform of a positive electrical stimulation signal, and FIG. 10C shows a schematic waveform of a negative electrical stimulation signal. Hereinafter, FIGS. 10A, 10B, and 10C will be described together with reference to FIG. 9 to help understanding of description.

Referring to FIG. 10A, 10B, and 10C, in an interval of t0 to t1, the first ultrasonic wave generating and transmitting element 320 and the second ultrasonic wave generating and transmitting element 330 may not generate an ultrasonic wave. Meanwhile, in an interval of t0 to t1, the electric stimulation controller 312 may generate a positive electrical stimulation signal and a negative electrical stimulation signal. For example, the positive electrical stimulation signal may be transmitted to the first ultrasonic wave generating and transmitting element 320, and the negative electrical stimulation signal may be transmitted to the second ultrasonic wave generating and transmitting element 330.

In an interval of t1 to t2, the first ultrasonic wave generating and transmitting element 320 and the second ultrasonic wave generating and transmitting element 330 may generate an ultrasonic wave. For example, the ultrasonic wave controller 311 may control the piezoelectric elements provided to the first ultrasonic wave generating and transmitting element 320 and the second ultrasonic wave generating and transmitting element 330, respectively, to generate an ultrasonic wave. Meanwhile, in the interval of t1 to t2, the electric stimulation controller 112 may not generate an electrical stimulation signal.

That is, the frequency of the ultrasonic wave may be a time between t0 to t2, and the ultrasonic wave may include an active interval and an inactive interval in one frequency. Likewise, that is, the frequency of the electrical stimulation signal may be a time between t0 to t2, and the electrical stimulation signal may include an active interval and an inactive interval in one frequency.

In an interval of t2 to t3, an interval of t3 to t4, and an interval of t4 to t5, an operation similar to the above may be implemented. That is, the positive electrical stimulation signal and the negative electrical stimulation signal may not be generated in the interval during which the ultrasonic wave is not generated, and the positive electrical stimulation signal and the negative electrical stimulation signal may be generated in the interval during which the ultrasonic wave is generated.

As described above, although the waveforms of the ultrasonic wave and the electrical stimulation signal, which are generated by the medical device according to an embodiment of the inventive concept, are schematically illustrated in FIG. 10A, 10B, and 10C, an embodiment of the inventive concept is not limited to the substantial waveform of the ultrasonic wave and the electrical stimulation signal. For example, the ultrasonic wave generated by the medical device according to an embodiment of the inventive concept may have a wave shaped waveform, and the electrical stimulation signal may have a shape of a pulse or an impulse. The waveforms illustrated in FIG. 10A, 10B, and 10C are intended to show that the ultrasonic wave and the electrical stimulation signal are alternately generated, and the positive electrical stimulation signal and the negative electrical stimulation signal are generated. However, an embodiment of the inventive concept is not limited to the embodiment in FIG. 10A, 10B, and 10C.

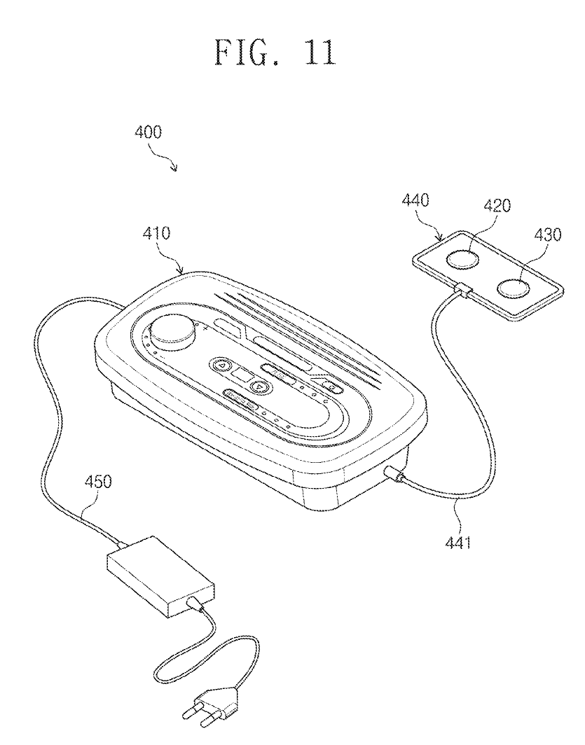

FIG. 11 is a view exemplarily illustrating a medical device according to an embodiment of the inventive concept. Referring to FIG. 11, a medical device 400 may include a controller 410, ultrasonic wave generating and transmitting elements 420 and 430, a probe 440, and a power cable 450.

The controller 410 may include the ultrasonic wave controller and the electric stimulation controller, which are described in this specification. The controller 410 may include a user interface (e.g., a button, a touch pad, etc.) for selecting the strength of the ultrasonic wave and the electrical stimulation signal and various operation modes.

The ultrasonic wave generating and transmitting elements 420 and 430 may include the functional material accommodation space (i.e., housing) that are previously described and maximize a surface area in which the functional material contacts the skin.

The ultrasonic wave generating and transmitting elements 420 and 430 may generate an ultrasonic wave under control of the ultrasonic wave controller provided in the controller 410. Also, the ultrasonic wave generating and transmitting elements 420 and 430 may transmit an electrical stimulation signal generated by the electric stimulation controller provided in the controller 410 to the skin. For example, the ultrasonic wave generating and transmitting elements 420 and 430 may generate the ultrasonic wave in the interval during which the electrical stimulation signal is not generated and may not generate the ultrasonic wave in the interval during which the electrical stimulation signal is generated. For example, the ultrasonic wave generating and transmitting element may generate a positive electrical stimulation signal, and the second ultrasonic wave generating and transmitting element 430 may generate a negative electrical stimulation signal.

Although the probe 440, for convenience of the user, has a pad shape including the ultrasonic wave generating and transmitting elements 420 and 430 as an example, an embodiment of the inventive concept is not limited to the shape of the probe 440. The probe 440 may be connected to the controller 410 through the probe cable 441 for being connected to the controller 410. The probe cable 441 may include a signal line connected to the piezoelectric element (e.g., 121 in FIG. 2) provided to the ultrasonic wave generating and transmitting elements 420 and 430, respectively, and a signal line connected to the transmission unit (e.g., 122 in FIG. 2) to electrically connect the controller 410 to the ultrasonic wave generating and transmitting elements 420 and 430.

The controller 410 may receive a power from the outside through the power cable 450. However, depending on an embodiment, the power cable 450 may not be provided. In this case, a separate battery module (not shown) for operating the medical device 400 may be provided.

As described above, various embodiments of the medical devices according to an embodiment of the inventive concept are described. According to the embodiments of the inventive concept, since the functional material accommodation space is defined by the inner side of the guide and the one surface of the ultrasonic wave generating and transmitting element, the surface area in which the functional material contacts the skin may be maximized. As a result, since it is minimized that the ultrasonic wave is reflected or distorted by metal, the functional material may be effectively injected into the skin.

In addition, the medical device according to the embodiment of the inventive concept generates the ultrasonic wave and the electrical stimulation signal in an alternate manner. That is, the electrical stimulation signal is not generated in the interval during which the ultrasonic wave is generated, and the electrical stimulation signal is generated in the interval during which the ultrasonic wave is not generated. As a result, the medicine injection effect caused by the ultrasonic wave and the physical treatment effect caused by the electrical stimulation signal may be achieved at the same time, and the circuit for generating the ultrasonic wave and the electrical stimulation signal may be simply configured.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

1. A medical device comprising:
a piezoelectric element configured to generate an ultrasonic wave;
a transmission unit configured to transmit the ultrasonic wave and an electrical stimulation signal generated from the outside to a skin, while contacting the piezoelectric element; and
a guide having a ring shape having an inner side and an outer side and contacting one surface of the transmission unit in order to define a functional material accommodation space,
wherein the functional material accommodation space is defined by the inner side of the guide and the one surface of the transmission unit.

2. The medical device of claim 1, wherein a functional material provided to the functional material accommodation space is injected into the skin by using the ultrasonic wave.

3. The medical device of claim 1, wherein the piezoelectric element generates the ultrasonic wave at an interval of a reference frequency, and
the piezoelectric element generates the ultrasonic wave in an interval during which the electrical stimulation signal is not generated.

4. The medical device of claim 1, further comprising:
an electric stimulation controller configured to generate the electrical stimulation signal; and
an ultrasonic wave controller configured to control the piezoelectric element so that the piezoelectric element generates the ultrasonic wave.

5. The medical device of claim 4, further comprising:
a first electrode film provided on a first surface of the piezoelectric element and connected to the ultrasonic wave controller; and
a second electrode film provided on a second surface of the piezoelectric element and connected to the ultrasonic wave controller.

6. The medical device of claim 1, wherein the ultrasonic wave generating and transmitting element comprises a conductive material.

7. The medical device of claim 1, wherein the piezoelectric element comprises at least one of lead zirconium titanate, barium titanate, ammonium dihydrogen phosphate, and ethylenediamine tartrate.

8. The medical device of claim 1, wherein the functional material accommodation space has a depth of about 0.05 mm to about 0.7 mm.

9. The medical device of claim 1, wherein the functional material accommodation space has a constant inner diameter along a depth direction.

10. A medical device comprising:
an electric stimulation controller configured to generate an electrical stimulation signal; and
an ultrasonic wave generating and transmitting element comprising a piezoelectric element configured to generate an ultrasonic wave under control of an ultrasonic wave controller and a housing defined in a depth direction to accommodate a functional material,
wherein the ultrasonic wave generating and transmitting element is configured to transfer the functional material provided in the housing to the skin by using the ultrasonic wave,
the ultrasonic wave generating and transmitting element is configured to transmit the electrical stimulation signal to the skin, and
the housing has a constant inner diameter along the depth direction.

11. The medical device of claim 10, wherein the ultrasonic wave is generated in an interval during which the electrical stimulation signal is not generated, and
the electrical stimulation signal is generated in an interval during which the ultrasonic wave is not generated.

12. The medical device of claim 10, further comprising:
a first electrode film provided on a first surface of the piezoelectric element and connected to the ultrasonic wave controller; and
a second electrode film provided on a second surface of the piezoelectric element and connected to the ultrasonic wave controller.

13. The medical device of claim 10, wherein the housing comprises a conductive material.

14. A medical device comprising:
an electric stimulation controller configured to generate a first electrical stimulation signal having a first polarity and a second electrical stimulation signal having a second polarity;
a first ultrasonic wave generating and transmitting element comprising a first piezoelectric element configured to generate an ultrasonic wave under control of an ultrasonic wave controller and a first housing defined in a depth direction to accommodate a functional material; and
a second ultrasonic wave generating and transmitting element comprising a second piezoelectric element configured to generate the ultrasonic wave under the control of the ultrasonic wave controller and a second housing defined in the depth direction to accommodate the functional material,
wherein the first ultrasonic wave generating and transmitting element and the second ultrasonic wave generating and transmitting element are configured to transmit the functional material provided in the first housing and the second housing to the skin in a respective manner,
the first ultrasonic wave generating and transmitting element is configured to transmit the first electrical stimulation signal to the skin through the first housing, and the second ultrasonic wave generating and transmitting element is configured to transmit the second electrical stimulation signal to the skin through the second housing, and
each of the first housing and the second housing has a constant inner diameter along the depth direction.

15. The medical device of claim 14, wherein each of the first piezoelectric element and the second piezoelectric element generates the ultrasonic wave at an interval of a reference frequency, and
the electric stimulation controller generates the first electrical stimulation signal and the second electrical stimulation signal in an interval during which the ultrasonic wave is not generated.

16. The medical device of claim 14, further comprising:
a first electrode film provided on a first surface of the first piezoelectric element and connected to the ultrasonic wave controller;
a second electrode film provided on a second surface of the first piezoelectric element and connected to the ultrasonic wave controller;
a third electrode film provided on a first surface of the second piezoelectric element and connected to the ultrasonic wave controller; and
a fourth electrode film provided on a second surface of the second piezoelectric element and connected to the ultrasonic wave controller.

17. The medical device of claim 14, wherein each of the first housing and the second housing has a depth of about 0.05 mm to about 2.0 mm.

18. The medical device of claim 14, wherein each of the first housing and the second housing comprises a conductive material.
